(19) United States
(12) Patent Application Publication
(10) Pub. No.: US 2005/0245585 A1
(43) Pub. Date: Nov. 3, 2005

(54) Process for preparing optically pure zolmitriptan

(76) Inventors: Islam Aminul, Dhubri (IN); Bhar Chandan, Hooghly District (IN); Katam Sahadev, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/111,372

(22) Filed: Apr. 21, 2005

Related U.S. Application Data
(60) Provisional application No. 60/604,166, filed on Aug. 24, 2004.

(30) Foreign Application Priority Data
Apr. 22, 2004 (IN) 368/CHE/2004

(51) Int. Cl. A61K 31/422; C07D 413/02

(52) U.S. Cl. 514/376; 548/229

(57) ABSTRACT

A process for preparing zolmitriptan, proceeding through the intermediate Ethyl-3-[2-(1,3-dioxo-2,3-dihydro-1H-2-isoindoleyl)ethyl]-5-[(4S)-2-oxo-1,3-oxazolan-4-ylmethyl]-1H-2-indole carboxylate.

### CROSS-REFERENCE TO RELATED APPLICATIONS

This application claims priority from copending U.S. Provisional Application 60/604,166 filed Aug. 24, 2004 and from India Patent Application 368/CHE/2004 filed Apr. 22, 2004. The entire content of each of the prior applications is hereby incorporated by this reference.

### INTRODUCTION TO THE INVENTION

The present invention relates to processes to prepare an optically pure pharmaceutical active compound, particularly the compound zolmitriptan. Zolmitriptan is known as (4S)-4-[3-(2-dimethyl amino ethyl)-1H-5-indolyl methyl]-1,3-oxazolan-2-one, or (S)-4-[[3-[2-(dimethylamino)ethyl]-1H-indol-5-yl]methyl]-2-oxazolidinone, has the formula C₁₆H₂₁N₃O₂ and a molecular weight of 287.36, and is represented by structure (I):

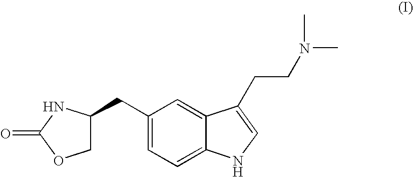

The present invention also relates to a process of purification of optically pure zolmitriptan.

Zolmitriptan is a dual action 5-HT 1B/1D receptor agonist that is used as a therapeutic agent for treating migraine. Nasal spray and tablet products containing the drug are sold using the trademark ZOMIG.

Earlier disclosures for the preparation of optically pure zolmitriptan have disadvantages, particularly for scale-up to produce commercial quantities.

International Patent Publication WO 91/18897 of The Wellcome Foundation Limited disclosed the preparation of zolmitriptan by diazotization of (4S)-4-(4-aminobenzyl)-1,3-oxazolan-2-one followed by reduction to form the corresponding hydrazine intermediate and coupling with 1-butyraldehyde (or its acetal) substituted at C4 with an easy leaving group (such as chlorine or protected amine) by Fischer reaction. The resultant compound was converted to (4S)-4-[3-(2-dimethyl amino ethyl)-1H-5-indolyl methyl]-1,3-oxazolan-2-one by standard N-alkylation methods. The disadvantages lie in utilizing the Fischer reaction for further scale-up, as the aldehydes are volatile and unstable in nature.

International Patent Publication WO 97/06162 of The Wellcome Foundation Limited discloses the preparation of zolmitriptan by diazotization of (4S)-4-(4-aminobenzyl)-1,3-oxazolan-2-one, followed by reduction to form the corresponding hydrazine intermediate and coupling with 4,4-methoxy-N,N-dimethylbutylamine by Fischer reaction. The disadvantages lie in high dilution of the reaction mass, extraction at elevated temperature, etc.

International Patent Publication WO 01/34561 of Knoll Aktiengesellschaft describes preparations of intermediate compounds, for use in preparing zolmitriptan. The preparation involves a Fischer indole synthesis.

International Patent Publication WO 2004/014901 of Laboratorios Vita, S.A. describes another process for preparing zolmitriptan. But like earlier processes, this patent also uses a process involving the Fischer reaction. Also, the other part of the zolmitriptan moiety is prepared from α-keto-δ-valerolactone that is commonly used chemical industrial purposes. Also optical purity is not disclosed. Strong alkali reagents are used in the hydrolysis stage that may open the oxazolidinone ring when following the described process. Also, we found the decarboxylation process is more complicated in terms of the reagents and conditions used in the process disclosed in this publication.

There remains a need for a process that is easily conducted on a commercial scale, to produce a product having a high purity.

### SUMMARY OF THE INVENTION

A first aspect of the present invention comprises a process for the preparation of zolmitriptan by the steps:

(a) forming a diazo salt having formula (IV) from (4S)-4-(4-aminobenzyl)-1,3-oxazolan-2-one, 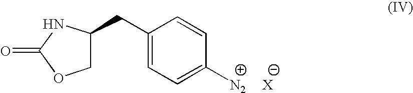
where X represents a halogen;

(b) forming an enol derivative having formula (V) from alkyl-2-acetyl-5-(1,3-dioxo-2,3-dihydro-1H-2-isoindolyl) pentanoate, 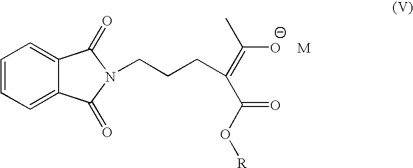
where R represents a straight or branched chain C₁-C₄ alkyl group and M represents an alkali metal;

(c) coupling diazo salt (IV) and enol derivative (V) to form a hydrazone having formula (VI) 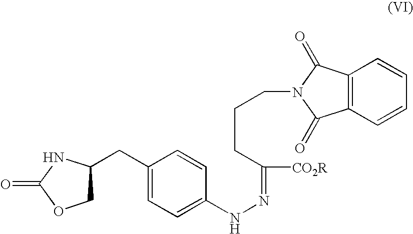
where R represents a straight or branched chain C₁-C₄ alkyl group;

(d) a cyclization of hydrazone (VI) to form an indole derivative having formula (VII) 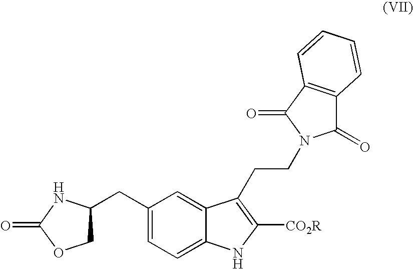
where R represents a straight or branched chain C₁-C₄ alkyl group;

(e) deprotecing the phthalimide moiety of (VII) to form a primary amine having formula (VIII), 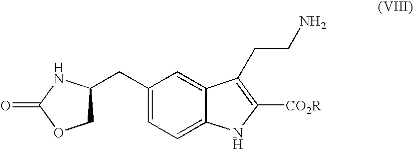
where R represents a straight or branched chain C₁-C₄ alkyl group;

(f) N,N-dimethylating primary amine (VII) to form a tertiary amine having formula (IX), 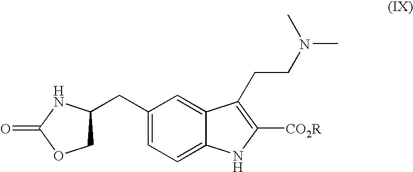
where R represents a straight or branched chain C₁-C₄ alkyl group; and

(g) decarboxylating tertiary amine (IX) to form zolmitriptan.

A second aspect of the present invention comprises a process for the preparation of zolmitriptan by the steps:

(i) hydrolyzing an an indole derivative having formula (VII), 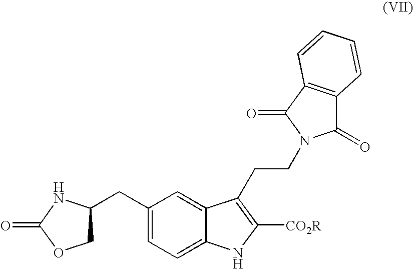
to form an acid having formula (X),

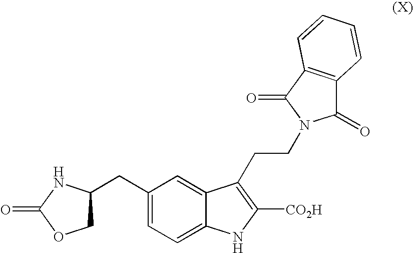

(ii) decarboxylating the acid (X) to form an isoindoledione having formula (XI),

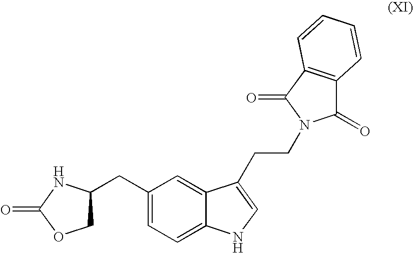

(iii) deprotecting the phthalimide moiety of the isoindoledione (XI) to form an oxazolan-2-one having formula (XII), 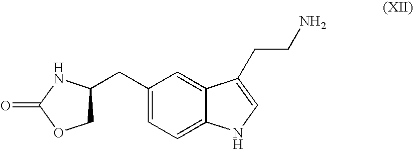
and

(iv) N,N-dimethylating the oxazolan-2-one (XII) to form zolmitriptan.

Another aspect of the present invention is providing non solvated, pure zolmitriptan.

In a further aspect, the present invention provides compounds having the following structures (VI), (VII), (VIII), (X) and (XI).

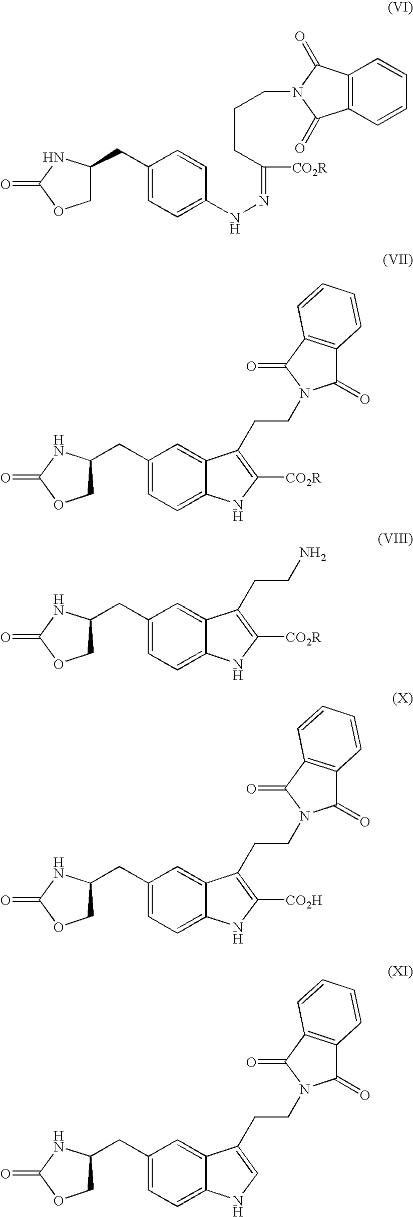

In a still further aspect, the invention provides processes for the preparation of compounds having the structures (VI) to (XI).

### BRIEF DESCRIPTION OF THE DRAWING

FIG. 1 is a diagram of a process scheme for preparing zolmitriptan.

### DETAILED DESCRIPTION

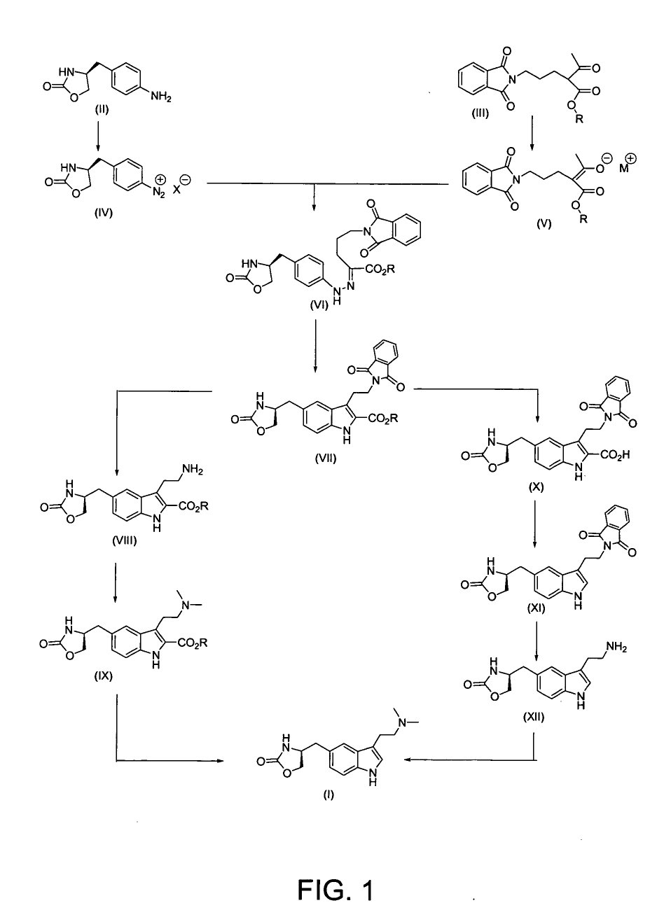

A first aspect of present invention includes a process for the preparation of zolmitriptan with reference to the scheme shown in FIG. 1, comprising the steps of:

(a) diazotizing (4S)-4-(4-aminobenzyl)-1,3-oxazolan-2-one (described in U.S. Pat. No. 6,303,791 to Patel) of the formula (II) 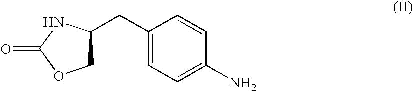
to form the corresponding diazo salt (IV), where the X in the structural formula is a halogen, and

(b) separately forming the enolate derivative (V) of alkyl-2-acetyl-5-(1,3-dioxo-2,3-dihydro-1H-2-isoindolyl) pentanoate (described in Khimiya Geterotsiklicheskikh Soedinenii, Vol. 11, pp. 1496-1501, 1974) of the formula (III) 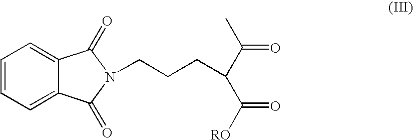
where R represents a C₁-C₄ alkyl group of either straight or branched chain and M in the structural formula (V) is an alkali metal,

(c) coupling (IV) and (V), such as in a neutral or mildly acidic medium via a Japp-Klihgemann reaction, to form the hydrazone of the formula (VI)

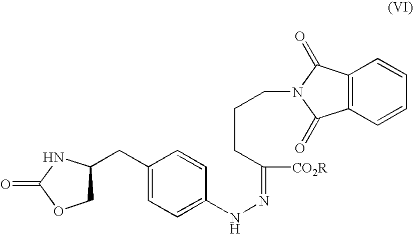

(d) cyclizing the hydrazone of the formula (VI) to the indole derivative alkyl-3-[2-(1,3-dioxo-2,3,3a,7a-tetrahydro-1H-2-isoindolyl)ethyl]-5-[(4S)-2-oxo-1,3-oxazolan-4-yl methyl]-1H-2-indole carboxylate of the formula (VII) 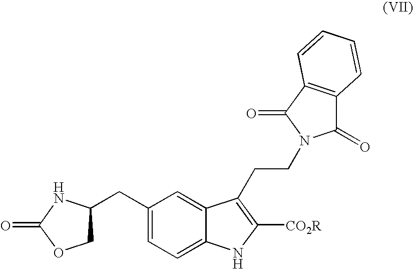
where R represents C₁-C₄ alkyl group of either straight or branched chain.

The process continues from this compound VII, by:

(1) deprotecting the phthalimide moiety of (VII) to give rise to the corresponding primary amine derivative alkyl 3-(2-aminoethyl)-5-[(4S)-2-oxo-1,3-oxazolan-4-yl methyl]-1H-2-indole carboxylate of the formula (VIII) and/or amine salts thereof 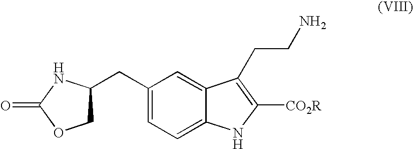
where R represents a C₁-C₄ alkyl group of either a straight or branched chain; then

(2) N,N-dimethylating the primary amine (VIII) to synthesize the tertiary amine of the formula (IX) 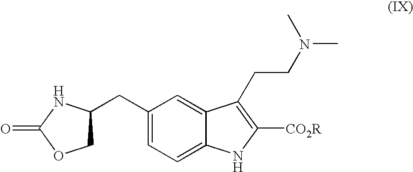
where R represents C₁-C₄ alkyl group of either straight or branched chain; and.

(3) treating the ester (IX) with an aqueous solution of a mild base such as sodium carbonate or potassium carbonate followed by treating with a strong acid such as concentrated HCl, to give zolmitriptan (I).

As an alternative, beginning with alkyl-3-[2-(1,3-dioxo-2,3,3a,7a-tetrahydro-1H-2-isoindolyl)ethyl]-5-[(4S)-2-oxo-1,3-oxazolan-4-yl methyl]-1H-2-indole carboxylate (VII), the process to form zolmitriptan comprises, in sequence:

(1a) hydrolyzing the ester group of the indole derivative of the formula (VII) to the corresponding carboxylic acid of the formula (X)

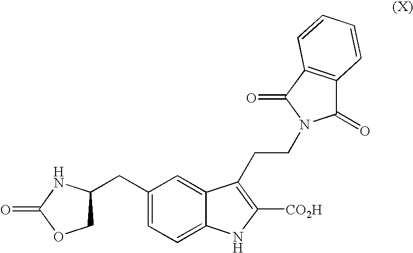

(2a) decarboxylating (X) in the presence of an acid and at an elevated temperature to form 2-(2-{5-[(4S)-2-oxo-1,3-oxazolan-4-yl-methyl]-1H-3-indolyl}ethyl)-2,3,3a,7a-tetrahydro-1H-1,3-isoindoledione of the formula (XI)

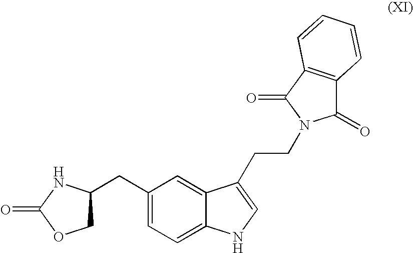

(3a) deprotecting the phthalimide moiety of (XI) to form (4S)-4-[3-(2-aminoethyl)-1H-5-indolylmethyl]-1,3-oxazolan-2-one of the formula (XII)

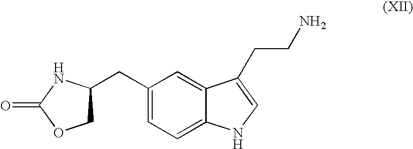

and

(4a) the N,N-dimethylation of (XII) to form zolmitriptan (I) or pharmaceutically acceptable salts thereof.

The hydrazone intermediate of the formula (VI) does not have to be isolated. This compound (VI) can optionally be converted in situ to the indole derivative alkyl-3-[2-(1,3-dioxo-2,3,3a,7a-tetrahydro-1H-2-isoindolyl)ethyl]-5-[(4S)-2-oxo-1,3-oxazolan-4-yl methyl]-1H-2-indole carboxylate of the formula (VII), through an acid catalyzed 3,3-sigmatropic shift in a moisture controlled medium. This cyclization step can be carried out in an alcoholic medium such as CₙH₂ₙ₊₁OH, where n=1-4, either straight or branched, having free H⁺ dissolved therein (e.g., HCl) and could be affected by the presence of excess moisture (e.g., >20%) and also by low concentrations of dissolved H⁺ (e.g., <5%).

The alkyl group R of compounds (VI), (VII), (VIII), and (IX) can be a single group like methyl, ethyl, isopropyl, etc. or may be a mixture of such groups depending on the R group in compound (III) and the alkyl group of the alcohol in the alcoholic solution used as the medium, as well as the reagent used for cyclization of the hydrazone intermediate (VI). In the case where both alkyl groups are the same, e.g., R=C₂H₅ in the starting compound (III) and C₂H₅OH—HCl is used in the cyclization in step (b), all of the intermediates (VI), (VII), (VIII), and (IX) contain a single R group (e.g.: R=C₂H₅). In the case where the alkyl groups are different, e.g.: R=C₂H₅ in the intermediate (IV) and CH₃OH—HCl is used in the cyclization step (b), the intermediates will be mixtures of two esters, e.g., R=mixtures of C₂H₅ and CH₃ in the intermediates (VI), (VII), (VIII), and (IX). The mutual ratio between the two esters can vary and is not important, due to the final decarboxylation to afford the zolmitriptan compound (I). In general, the ratio of the two alkyl groups in all of intermediates (VI), (VII), (VIII), and (IX). will be between 5:95 and 95:5, or between 10:90 and 90:10.

Step (1) prepares compound (VIII) by the Ing-Manaske reaction where phthalimide is deprotected using base such as an N-containing base like hydrazine or alkylamines (RNH₂). The reaction can be carried out in an alcohol such as CₙH₂ₙ₊₁OH, where n=1-4, both straight and branched, and/or in halogenated solvents (CHₙX₄₋ₙ where each X is independently a halogen atom). The formed amine of the formula (VIII) can be isolated both in the free amine form as well as in the corresponding salt form. Maintaining the intermediate in its salt form is preferable from a process point of view, as compound (VIII) tends to cyclize to form the cyclized amide of formula (XIII) after a prolonged maintenance in a basic medium:

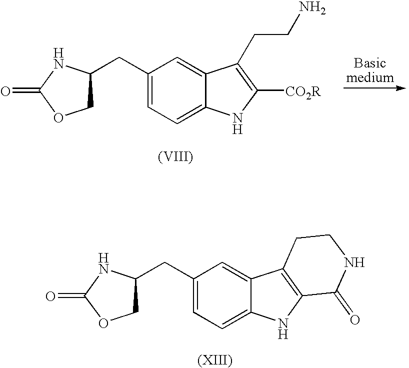

Step (2) is an Esweiler-Clark methylation of intermediate (VIII) to prepare the compound of formula (IX). Due to the complex nature of compound (IX), a comparatively low-temperature reaction is chosen where a metal hydride (e.g., sodium cyanoborohydride, or sodium borohydride) is employed as the hydrogen donor while maintaining the pH in a mildly acidic region, such as using acetic acid buffer systems. It was found that the reaction yields an impure reaction mass when formic acid was chosen as the hydrogen donor and the reaction was carried out at an elevated temperature (>150° C.). Formaldehyde in the form of an aqueous solution or as paraformaldehyde can be used as the methylating reagent.

Step (3) is a one-pot reaction wherein the intermediate (IX) is first treated with a non-hydroxyl base in an aqueous medium, followed by the treatment with strong aqueous acid to afford zolmitriptan of the formula (I). A relatively milder base (such as sodium carbonate or potassium carbonate) is chosen since stronger alkalies such as sodium, potassium, or barium hydroxides lead to the rupture of the oxazolidinone structure.

In another aspect, zolmitriptan can be prepared from intermediate (VII) following the sequence of steps (1), (2), and (3) without isolating the intermediate (IX). The alkyl 3-(2-aminoethyl)-5-[(4S)-2-oxo-1,3-oxazolan-4-yl methyl]-1H-2-indole carboxylate of formula (VIII), obtained from step-(1) is subjected to N,N-dimethylation following the same Eschweiler-Clark procedure stated above. The product of this reaction, alkyl 3-(2-dimethyl amino ethyl)-5-[(4S)-2-oxo-1,3-oxazolan-4-yl-methyl]-1H-2-indole carboxylate of formula (IX), is treated in-situ with mild non-hydroxyl bases in an aqueous medium, followed by aqueous acid treatment, in step 3, to afford the zolmitriptan product.

Using a different synthetic approach, in step (1a), the compound (VII) is hydrolyzed to afford the acid (X). The intermediate (X) is then decarboxylated in step (2a) in strong acid medium to afford the intermediate (XI). The intermediate (X) and the decarboxylated intermediate (XI) optionally may not be isolated after they are formed.

The in situ intermediate (XI) is then subjected to deprotection of its phthalamide moiety in step (3a) to prepare compound of formula (XII).

N,N-dimethylation of compound of formula (XII) in step (4a) affords the zomitriptan product.

According to an aspect of present invention, there is provided a process for the purification of optically pure (4S)-4-[3-(2-dimethyl amino ethyl)-1H-5-indolyl methyl]-1,3-oxazolan-2-one which comprises:
(i) dissolving the crude (4S)-4-[3-(2-dimethyl amino ethyl)-1H-5-indolyl methyl]-1,3-oxazolan-2-one (I) in isopropyl alcohol at an elevated temperature and filtering the hot solution under positive pressure.
(ii) slowly cooling the filtrate to a temperature of 20-30° C.
(iii) adding n-Heptane to the mixture in a dropwise manner under stirring at 20-30° C. for 15-20 minutes.
(iv) filtration, or, centrifugation of the solid product obtained from the step (iii).
(v) washing the cake with n-heptane to remove solvated isopropanol.
(vi) drying of the cake.

For recrystallization of zolmitriptan, the use of relatively less polar alcohols (e.g.: isopropyl alcohol) as a single solvent and/or in conjugation with an almost non-polar hydrocarbon solvent (e.g., n-heptane, n-hexane, petroleum ether) is found to achieve a consistent result in achieving a high purity level and simultaneously reducing the impurity level to that defined by international guidelines for an active pharmaceutical ingredient. Particularly when it is desired to reduce the desmethyl impurity of zolmitriptan to a very low level, steps (i) through (v) of the recrystallization procedure will be repeated several times.

The preferable quantity of isopropanol in the step (i) is three times weight-to-volume ratio against the crude compound having a purity of 95-99%. The hot solution can be decolorized using decolorizing charcoal prior to the filtration or centrifugation.

The preferable quantity of n-heptane in the step (iii) is a weight-to-volume ratio of one, versus the crude compound of purity 95-99%.

For complete transfer of the solid to the filtration bed a mixture of isopropyl alcohol and n-heptane can be used. The preferable composition is a 50:50 mixture, cooled to 10-15° C. before use.

The drying stage of step (vi) can be carried out at an elevated but non-extreme temperature under vacuum. The preferable range of temperature employed is 60-65° C. and the vacuum is 50-100 mBar.

The resulting product is a non-solvated solid of high chemical and optical purity to qualify as an active pharmaceutical ingredient under international standards.

In another aspect, the present invention provides non-solvated, pure (4S)-4-[3-(2-dimethyl amino ethyl)-1H-5-indolyl methyl]-1,3-oxazolan-2-one.

In a further aspect, the present invention provides compounds of the following formulae (VI), (VII), (VIII), (X), and (XI).

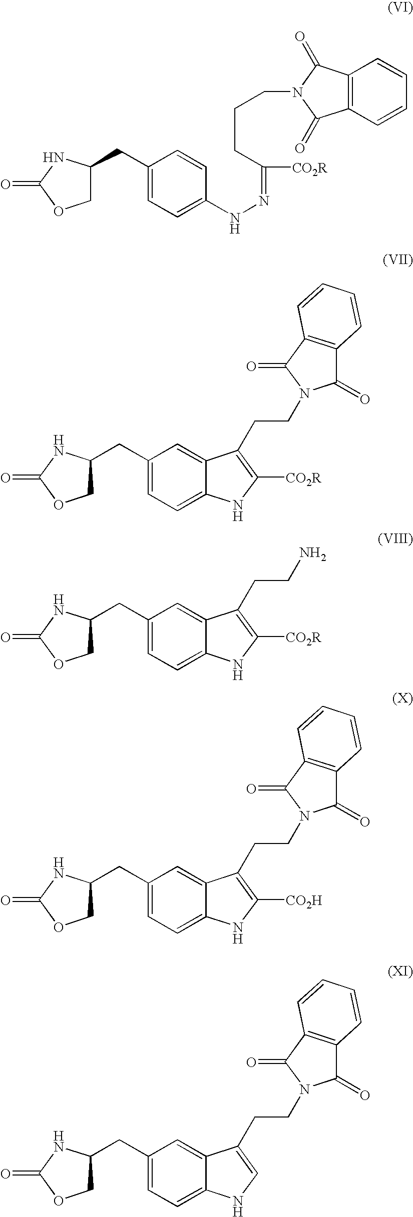

In a still further aspect, the invention provides processes for the preparation of compounds (IV) to (XII). For compounds (IV) to (VI) see process steps (a), (b), and (c) that were previously described. For compound (VII) see process step (d) that was previously described. For compound (VIII) see process step (1) as previously described. For compound (IX) see process step (2) as previously described. The preparation of compound (X) is shown by process step (1a) described above. Compound (XI) is prepared by process step (2a) in the prior description. For the preparation of compound (XII) see step (3a) in the prior description.

The aforesaid compounds of formula (IV) through (XII) are useful as intermediates for the synthesis of zolmitriptan, and their use for the synthesis of other products likewise forms part of the scope of the present invention.

Among the advantages of the invention are the following:
a. Use of relatively low cost and easily available raw materials. The entire process is cost effective.
b. A simple scalable process that enables one having ordinary skilled in the art to achieve a consistent result within the specified limit.
c. The process gives a robust process to furnish the chemically and optically pure zolmitriptan according to strict international standards for active pharmaceutical ingredient with a good quality management.

Unless stated otherwise, all technical and scientific terms used herein have the same meaning as commonly understood by one ordinary skilled in the art.

The invention is described in the examples given below, which are provided by way of illustration only and therefore should not be construed to limit the scope of the invention as it is defined in the claims.

### EXAMPLE 1

### Ethyl-3-[2-(1,3-dioxo-2,3-dihydro-1H-2-isoindoleyl)ethyl]-5-[(4S)-2-oxo-1,3-oxazolan-4-ylmethyl]-1H-2-indole carboxylate (VII)

Part A:

(4S)-4-(4-aminobenzyl)-1,3-oxazolan-2-one of the formula (II) (200 g, 1.04 moles) was dissolved in methanol (600 ml) and water (900 ml) and cooled to 5-10° C., then concentrated HCl (272 ml) was added dropwise to avoid heat generation and stirring was continued for 10 minutes. To this mixture sodium nitrite (90 g, 1.25 moles) solution in water (600 ml) was added slowly at −5 to 0° C. After the addition, the solution was maintained under stirring at the same temperature for 30 minutes.

Part B:

In a separate vessel, ethyl-2-acetyl-5-(1,3-dioxo-2,3-dihydro-1H-2-isoindolyl) pentanoate of the formula (III) (346.4 g, 1.09 moles) was added to methanol (3400 ml), sodium acetate (340 g, 4.15 moles) was added, and the mixture was stirred at room temperature for one hour. After cooling to 0-5° C. the Part A solution was added slowly. Following the addition, the mixture was stirred for 2-3 hours at room temperature. Progress of the reaction was monitored by TLC using ethyl acetate as the mobile phase.

The reaction mass was extracted with dichloromethane (2.5 L×3 times). The combined dichloromethane layer was washed with water (1.25 L×2 times), dried and concentrated to produce a crude residue containing the hydrazone intermediate, and this residue was used for cyclization to form the indole moiety. The hydrazone intermediate had the following properties:

¹H NMR: (DMSO-d₆, 200 MHz): δ 1.26 (t, 3H), 1.64-1.92 (m, 4H), 2.15-2.44 (d, 3H), 2.84-3.12 (d, 1H), 3.61-3.86 (m, 2H), 4.13-4.34 (m, 2H), 4.51 (t, 1H), 5.09-5.38 (m, 1H), 7.04-7.29 (m, 3H), 7.62-7.91 (m, 5H). MS: m/z 479 (100%, M+1; Et ester).

The above residue (560 g) was added to 300 ml of methanol, heated to form a transferable mass and, under refluxing conditions, 1.2 L methanolic HCl (15%) was added to this. The reaction mixture was further refluxed for 6 hours. The reaction mass was then cooled to room temperature and the obtained solid was filtered and washed with methanol.

Product:

Net weight: 375g Yield: 78% Purity: 97.42% (R=C₂H₅ is 68.38% and R=CH₃ is 29.04%) Melting Range.: 215-220° C. IR (KBr): 3357 (br), 2940 (br), 1759 (br), 1707, 1544, 1466, 1440, 1396, 1248, 1026 cm⁻¹. ¹H NMR: (DMSO-d₆, 200 MHz): δ 1.35 (t, 3H), 2.6-2.9 (m, 2H), 3.3-3.4 (m, 2H), 3.75 (s, —OCH₃), 3.8-3.9 (m, 4H), 4.08 (m, 1H), 4.21 (q, 2H), 7.04 (d, 1H, J=8.4 Hz), 7.35 (d, 1H, J=8.4 Hz), 7.38 (s, 1H, J=8.4 Hz), 7.78 (br, 4H), 11.51 (br, 1H, exchangeable). ¹³C-NMR: (DMSO-d₆, 200 MHz): δ 14.215, 23.418, 38.348, 40.852, 51.526, 53.165, 60.357, 68.171, 112.673, 118.887, 119.130, 119.850, 122.923, 123.810, 124.076, 126.678, 127.642, 127.725, 131.572, 134.325, 135.319, 158.739, 161.698, 162.054, 167.737 MS: m/z 462 (100%, M+1; Et ester), 448 (M+1; Me ester)

### EXAMPLE 2

### Ethyl 3-(2-aminoethyl)-5-[(4S)-2-oxo-1,3-oxazolan-4-ylmethyl]-1H-2-indole carboxylate: Hydrochloride Salt (VIII)

Ethyl-3-[2-(1,3-dioxo-2,3-dihydro-1H-2-isoindoleyl)ethyl]-5-[(4S)-2-oxo-1,3-oxazolan-4-ylmethyl]-1H-2-indole carboxylate of the formula (VII) (100 g, 0.217 moles) was dissolved in 800 ml of methanol at room temperature with stirring, then 42.17 ml (43.4 g, 0.867 moles) of hydrazine hydrate were added dropwise over about 20-30 minutes, with stirring at 35-40° C. The mixture was maintained under stirring at the same temperature for 150 minutes, and analyzed by TLC using ethyl acetate as the mobile phase to show completion of the reaction.

After the reaction was completed, the reaction mass was cooled to 10-15° C. and 950 ml of 2N HCl were added to it in a dropwise manner. The mass was diluted with 550 ml of water, evaporated to remove methanol, and cooled to 10-15° C. After stirring, the solid was removed by filtration. The filtrate was concentrated to a 350-400 ml volume, cooled to 10-15° C. and 1000 ml dichloromethane were added, and stirring was continued for 15 minutes at 10-15° C. The resulting solid was filtered and recrystallized from a mixture of methanol (5 volumes) and acetone (20 volumes).

Product:

Net weight: 48.5 g Yield: 60.6% Purity: 97.76% (R=C₂H₅ is 85.28%, R=CH₃ is 12.48%). Melting Range: 260-265° C. IR (KBr): 3344, 3222, 3151, 2973 (br), 2933, 1761, 1691, 1594, 1542, 1470, 1442, 1338, 1252, 1034 cm⁻¹. ¹H-NMR (DMSO-d₆, 200 MHz): δ 1.37 (t, 3H), 2.88 (m, 2H), 3.04 (m, 2H), 3.38 (m, 2H), 3.84 (s, —OCH₃), 4.08 (m, 2H), 4.20-4.43 (m, 3H), 7.18 (d, 1H, J=8.4 Hz), 7.39 (d, 1H, J=8.4 Hz), 7.64 (s, 1H, J=8.4 Hz), 7.83 (s, 1H, exchangeable), 8.16 (br s, 2H, exchangeable), 11.49-11.67 (br, 1H, exchangeable). ¹³C-NMR (DMSO-d₆, 200 MHz): δ 14.382, 22.659, 40.852, 51.764, 52.930, 60.440, 68.270, 112.643, 117.635, 120.457, 124.152, 127.012, 127.391, 127.907, 135.342, 158.739, 161.561. MS (Electronspray, +ve mode): m/z 315.0 [(M+1)⁺ for Me ester], 332.0[(M+1)⁺ for Et ester, 100%), 663.3 [(2M+1)⁺ for Et ester]. MS (Electronspray, −ve mode): m/z 330.3 [(M−1)⁻, Et ester], 366.0 [(M−1+HCl)⁺ for Et ester; 100%].

### EXAMPLE 3

### Ethyl 3-(2-dimethyl aminoethyl)-5-[(4S)-2-oxo-1,3-oxazolan-4-yl methyl]-1H-2-indole carboxylate: Hydrochloride Salt (IX)

100 g (0.27 moles) of ethyl 3-(2-aminoethyl)-5-[(4S)-2-oxo-1,3-oxazolan4-ylmethyl]-1H-2-indole carboxylate hydrochloride salt (VIII) were dissolved in 1334 ml of methanol at room temperature under stirring, neutralized to pH 7 by adding 20% sodium carbonate solution dropwise, and 39.92 ml (40.8 g, 0.68 moles) of glacial acetic acid were added to it at the same temperature. The reaction mass was cooled to 10-15° C. Sodium cyanoborohydride (42.16 g, 0.68 moles) is added pinch by pinch to the reaction mixture with stirring at this temperature. After addition, the mixture was cooled to −5° to 0° C. A mixture of 209.2 ml of 38% aqueous formaldehyde solution (81.6 g, 2.72 moles) and 666 ml methanol was added to the reaction mixture in a dropwise fashion at −5 to 0° C., with stirring. Stirring was continued at the same temperature for another 60-90 minutes and then reaction progess was monitored by TLC using 30% methanol and 1.5% NH₃ in dichloromethane as the mobile phase. TLC showed the completion of reaction as the starting material disappeared.

To the reaction mixture, 286 ml of 20% (w/v) sodium carbonate solution is added at ˜0° C. and the mixture was then extracted with 2000 ml×3 times of dichloromethane. All dichloromethane layers were combined, washed with water followed by brine solution, dried over sodium sulfate and concentrated to 450-500 ml volume. The dichloromethane layer was then cooled to 10° C. and 443 ml of 1N hydrochloric acid (11.83 g, 0.32 moles) were added dropwise under stirring. The pH of the medium was finally adjusted to 5.5-6.0 by a dropwise addition of 20% sodium carbonate solution. The resulted solid was then filtered and dried.

Product:

Net weight: 67.5 g Yield: 63.2% Purity: 96.8% (R=C₂H₅is 80.4%, R=CH₃ is 16.4%) Melting Range: 195-200° C. IR (KBr): 3467, 3404, 3279, 2967 (br), 2683, 2634, 2477, 1739, 1723, 1699, 1547, 1471 (br), 1416, 1381, 1335, 1252, 1023 cm⁻¹. ¹H-NMR (DMSO-d₆, 200 MHz): δ 1.39 (t, 3H), 2.62-3.0 (m, 8H), 3.18-3.27 (br, 2H), 3.42-3.48 (br, 2H), 3.86 (s, —OCH₃), 3.89-4.19 (m, 2H), 4.21-4.42 (m, 2H), 7.19 (d, 1H, J=8.4 Hz), 7.40 (d, 1H, J=8.4 Hz), 7.67 (s, 1H, J=8.4 Hz), 7.84 (s, 1H, exchangeable), 10.84 (br, 1H, exchangeable), 11.62-11.79 (br, 1H, exchangeable). ¹³C-NMR (DMSO-d₆, 200 MHz): δ 14.435, 19.852, 38.348, 51.905, 52.793, 56.412, 60.675, 68.239, 112.719, 117.013, 120.685, 124.061, 127.133, 127.308, 127.922, 135.448, 158.891, 161.599, 162.009 (Me-ester). MS (Electronspray, +ve mode): m/z 360.0 [(M+1)⁺; Et ester, 100%], [346.1 (M+1)⁺; Me ester] MS (Electronspray, −ve mode): m/z 358.5 [(M−H)⁻, Et ester, 100%], 394.4 (M+HCl)⁻, 430.3 (M+2HCl)⁻, 753.7 (2M+HCl)⁻, 789.7 (2M+2HCl)⁻

### EXAMPLE 4

### (4S)-4-[3-(2-dimethyl amino ethyl)-1H-5-indolyl methyl]-1,3-oxazolan-2-one (I)

To 50 g (0.126 moles) of ethyl 3-(2-dimethyl aminoethyl)-5-[(4S)-2-oxo-1,3-oxazolan-4-yl methyl]-1H-2-indole carboxylate hydrochloride salt (IX), a solution of 26.88 g (0.253 moles) of sodium carbonate in 500 ml water was added under stirring. The reaction mixture was gently heated to reflux and monitored by TLC using 30% Methanol in dichloromethane as the mobile phase. After 2-2.5 hours of reflux, TLC showed a completion of reaction since the starting material vanished. Heating was discontinued and the mixture was allowed to cool to 15-18° C. A hydrolysis sample was kept for TLC monitoring and to the mixture 150 ml of conc. (35%) hydrochloric acid was added dropwise. The mixture was then heated to reflux and monitored by TLC using 30% Methanol and 1.5% NH₃ in dichloromethane. After 3 hrs of reflux TLC indicated completion.

The reaction mixture was cooled to 20-25° C. and extracted with 500 ml of ethyl acetate. The organic layer was discarded and the aqueous layer was further cooled to 15-20° C. 20% sodium hydroxide solution was added at this temperature to obtain pH 9.75-10. The mixture was extracted with ethyl acetate (1000 ml×3 times) at 25-30° C. and all ethyl acetate layers were mixed and washed with water (100 ml×2 times), then concentrated to 100-125 ml volume. After cooling to 25-30° C., 500 ml of n-heptane were added dropwise with stirring. After 20 minutes of stirring the solid was filtered, washed with n-heptane (100 ml) and dried.

Product:

Net weight: 28 g Yield: 77% Purity: 93.02% M.P. 132-135° C. ¹H-NMR (DMSO-d₆, 200 MHz): δ 2.22 (s, 6H), 2.52 (m, 2H), 2.78 (m, 1H), 2.80 (m, 2H), 2.89 (dd, J=4.8, 13.6 Hz, 1H), 4.0 (m, 1H), 4.05 (m, 1H), 4.23 (t, 1H, J=7.2 Hz), 6.90 (d, 1H, J=8.4 Hz), 7.10 (d, 1H, J=2.0 Hz), 7.25 (d, 1H, J=8.4 Hz), 7.75 (s, 1H), 10.70 (br, 1H). ¹³C-NMR (DMSO-d₆, 200 MHz): δ 23.09, 40.67, 45.12, 53.23, 59.99, 68.12, 111.26, 112.50, 118.75, 122.50, 122.69, 125.07, 127.57, 135.24, 158.75. MS: m/z 288 (M+1, 100%).

### EXAMPLE 5

### (4S)4-[3-(2-dimethyl amino ethyl)-1H-5-indolyl methyl]-1,3-oxazolan-2-one (I) (Via In Situ Intermediate IX)

25 g (0.068 moles) of ethyl 3-(2-aminoethyl)-5-[(4S)-2-oxo-1,3-oxazolan-4-ylmethyl]-1H-2-indole carboxylate hydrochloride salt (VIII) was dissolved in 250 ml methanol at room temperature with stirring, neutralized to pH 7 by adding 20% sodium carbonate solution dropwise, and 9.37 ml (10.2 g, 0.17 moles) of glacial acetic acid were added at the same temperature. The reaction mass was cooled to 10-15° C. Sodium cyanoborohydride (12.65 g, 0.204 moles) was added pinch by pinch to the mixture under stirring at this temperature. After addition, the mixture was cooled to −5° to 0° C. A mixture of 52.3 ml 38% aqueous solution of formaldehyde (20.4 g, 0.68 moles) and 75 ml methanol was added to the mixture in a dropwise fashion at −5 to 0° C., under stirring, then the stirring was continued at the same temperature for another 60-90 minutes. The reaction was monitored by TLC using 30% methanol and 1.5% NH₃ in dichloromethane as the mobile phase. TLC showed the completion of reaction when the starting material disappeared.

To the reaction mixture, 72.08 ml of 20% (w/v) sodium carbonate solution was added at about 0° C. and the mixture was then extracted with 375 ml×3 times of dichloromethane. All dichloromethane layers were combined, washed with water followed by brine solution and evaporated to dryness to yield 23 g of crude ethyl 3-(2-dimethyl aminoethyl)-5-[(4S)-2-oxo-1,3-oxazolan-4-yl methyl]-1H-2-indole carboxylate.

To the above crude product a solution of 10.1 g (0.096 mole) sodium carbonate in 230 ml water was added and refluxed under stirring. After 2-2.5 hours of reflux, TLC showed a completion of reaction since the starting material had vanished. Heating was discontinued and the mixture was allowed to cool to 15-18° C. A hydrolysis sample was kept for TLC monitoring and to the mixture 69 ml of concentrated (35%) hydrochloric acid was added dropwise. The mixture was then heated to reflux and monitored by TLC using 30% methanol & 1.5% NH₃ in dichloromethane. After 4 hours of reflux, TLC indicated completion.

The reaction mixture was cooled to 20-25° C. and extracted with 250 ml of ethyl acetate. The organic layer was discarded and the aqueous layer was further cooled to 15-20° C. and adjusted with 20% sodium hydroxide solution at this temperature to pH 9.75-10. Then the reaction mass was extracted with ethyl acetate (500 ml×3 times) at 25-30° C. and all ethyl acetate layers were mixed and washed with water (50 ml×2 times) and evaporated to dryness to yield 12.5 g residue. To the solid residue 37.5 ml of isopropyl alcohol was added and slowly heated to reflux to result in a clear solution. After slowly cooling to 20-30° C., 12.5 ml of n-heptane were added dropwise. The mixture was stirred for 15-20 minutes and then filtered. 12.5 ml of a prechilled 50:50 mixture of isopropanol and n-heptane were used to wash the reaction flask for a complete transfer of the solid. The cake was washed with 25 ml of n-heptane and dried at 50-55° C. under 50-100 mBar vacuum.

Result:

Net weight: 8.9 g Yield: 45% Purity: 98.55% M.P. 138-139° C. IR (KBr): 3240, 2920, 2856, 2776, 1742, 1479, 1443, 1403, 1242, 1069, 1030 cm⁻¹. ¹H-NMR (DMSO-d₆, 200 MHz): δ 2.22 (s, 6H), 2.52 (m, 2H), 2.78 (m, 1H), 2.80 (m, 2H), 2.89 (dd, J=4.8, 13.6 Hz, 1H), 4.0 (m, 1H), 4.05 (m, 1H), 4.23 (t, 1H, J=7.2 Hz), 6.90 (d, 1H, J=8.4 Hz), 7.10 (d, 1H, J=2.0 Hz), 7.25 (d, 1H, J=8.4 Hz), 7.75 (s, 1H), 10.70 (br, 1H). ¹³C-NMR (DMSO-d₆, 200 MHz): δ 23.09, 40.67, 45.12, 53.23, 59.99, 68.12, 111.26, 112.50, 118.75, 122.50, 122.69, 125.07, 127.57, 135.24, 158.75. MS: m/z 288 (M+1, 100%).

### EXAMPLE 6

### Purification of (4S)-4-[3-(2-dimethyl amino ethyl)-1H-5-indolyl methyl]-1,3-oxazolan-2-one (I)

To a crude ziprasidone product (8.8 g) 26.4 ml of isopropyl alcohol were added and slowly heated to reflux to result in a clear solution. The clear solution was treated with 1 g of decolorizing charcoal at refluxing conditions for 10-15 minutes and then filtered under a positive pressure while hot. The clear filtrate was then slowly cooled to 20-30° C. and 8.8 ml of n-heptane were added dropwise. Stirring continued for 15-20 minutes and the solution was filtered. 8.8 ml of a prechilled 50:50 mixture of isopropanol and n-heptane were used to wash the reaction flask and to transfer the solid completely. The cake was washed with 40 ml of n-heptane, then dried at 60-65° C. under 50-100 mBar vacuum until the moisture content and loss on drying became <1%.

Product:

Net weight: 8.2 g Yield: 93% Purity (Chemical): 99.7% Purity (Optical): 99.94% (other isomer 0.06%) IR (KBr): 3240, 2920, 2854, 2772, 1744, 1480, 1441, 1405, 1241, 1070, 1031 cm⁻¹. ¹H-NMR (DMSO-d₆, 200 MHz): δ 2.22 (s, 6H), 2.52 (m, 2H), 2.78 (m, 1H), 2.80 (m, 2H), 2.89 (dd, J=4.8, 13.6 Hz, 1H), 4.0 (m, 1H), 4.05 (m, 1H), 4.23 (t, 1H, J=7.2 Hz), 6.90 (d, 1H, J=8.4 Hz), 7.10 (d, 1H, J=2.0 Hz), 7.25 (d, 1H, J=8.4 Hz), 7.75 (s, 1H), 10.70 (br, 1H). ¹³C-NMR (DMSO-d₆, 200 MHz): δ 23.09, 40.67, 45.12, 53.23, 59.99, 68.12, 111.26, 112.50, 118.75, 122.50, 122.69, 125.07, 127.57, 135.24, 158.75. MS (Electronspray): m/z 288.1 (M+1)⁺, 310.3 (M+Na)⁺, 575.5 (2M+1)⁺, 597.5 (2M+Na)⁺.

### EXAMPLE 7

### (4S)-4-[3-(2-aminoethyl)-1H-5-indolylmethyl]-1,3-oxazolan-2-one (XII)

A solution of potassium carbonate (4.66 g, 0.033 mole) in water (20 ml) was added to 3.9 g (0.0084 mole) ethyl-3-[2-(1,3-dioxo-2,3-dihydro-1H-2-isoindoleyl)ethyl]-5-[(4S)-2-oxo-1,3-oxazolan-4-ylmethyl]-1H-2-indole carboxylate (VII) and the mixture was refluxed for 2 hours to obtain a clear solution. (At this stage the intermediate (X) can be isolated using the procedure described in Example 12 or processed further as in the following procedure).

The mixture was cooled to room temperature and concentrated HCl was added dropwise to pH 2; the mixture was refluxed for 5 hours. After cooling to room temperature, the mixture was extracted with ethyl acetate to remove organic impurities. the aqueous layer was adjusted with 20% NaOH solution to pH 10-11 and extracted with ethyl acetate. The ethyl acetate layer was dried over sodium sulfate and evaporated to dryness to yield a dark brown gummy crude mass that was used as such for further processing.

Product:

Net Weight: 0.91 g Yield: 41.5% IR: 3341 (br), 3142, 2962 (br), 1749, 1701, 1609, 1523, 1470, 1234, 983 cm⁻¹. ¹H NMR (200 MHz, DMSO-d₆): δ 2.56 (s, 2H), 2.78-2.9 (m, 5H), 4.02 (m, 2H), 4.25 (m, 1H), 6.91 (d, 1H, J=8.4 Hz), 7.16 (d, 1H, J=2.0 Hz), 7.27 (d, 1H, J=8.4 Hz), 7.43 (s, 1H), 7.83 (s, 1H), 8.38 (s, 1H) 10.80 (s, 1H). ¹³C-NMR (200 MHz, DMSO-d₆): δ 23.3, 39.8, 44.1, 54.4, 69.5, 110.2, 113.2, 119.8, 122.6, 126.2, 127.9, 130.1, 137.1, 158.2.

### EXAMPLE 8

### (4S)-4-[3-(2-dimethyl amino ethyl)-1H-5-indolyl methyl]-1,3-oxazolan-2-one (I)

The crude mixture from Example 7 (0.5 g, 0.0019 moles) was dissolved in methanol and sodium cyanoborohydride (0.23 g, 0.0036 moles) and acetic acid (0.3 ml) were added at 5-10° C. Formaldehyde (0.199 g =0.5 ml of 37% aqueous solution, 0.0066 moles) was mixed with methanol (3 ml) and added dropwise to the above mixture at 5-10° C. After this addition, the mixture was stirred at room temperature for 2 hours. TLC verified reaction completion, using 5% ammonia in methanol.

The mixture was adjusted with 10% potassium carbonate solution to pH 10-11 and then extracted with ethyl acetate several times. The ethyl acetate layers were combined, washed with brine and water and evaporated to produce a dark brown crude product.

Product:

Net Weight: 0.41 g Yield: 74.0% IR (KBr): 3239, 2915, 2850, 2776, 1747, 1477, 1445, 1403, 1245, 1070, 1031 cm⁻¹. ¹H-NMR (DMSO-d₆, 200 MHz): δ 2.22 (s, 6H), 2.52 (m, 2H), 2.78 (m, 1H), 2.80 (m, 2H), 2.89 (dd, J=4.8, 13.6 Hz, 1H), 4.0 (m, 1H), 4.05 (m, 1H), 4.23 (t, 1H, J=7.2 Hz), 6.90 (d, 1H, J=8.4 Hz), 7.10 (d, 1H, J=2.0 Hz), 7.25 (d, 1H, J=8.4 Hz), 7.75 (s, 1H), 10.70 (br, 1H). ¹³C-NMR (DMSO-d₆, 200 MHz): δ 23.09, 40.67, 45.12, 53.23, 59.99, 68.12, 111.26, 112.50, 118.75, 122.50, 122.69, 125.07, 127.57, 135.24, 158.75. MS: m/z 288 (M+1, 100%).

### EXAMPLE 9

### Ethyl-3-[2-(1,3-dioxo-2,3-dihydro-1H-2-isoindoleyl)ethyl]-5-[(4S)-2-oxo-1,3-oxazolan-4-ylmethyl]-1H-2-indole carboxylate (VII)

Part A:

(4S)-4-(4-aminobenzyl)-1,3-oxazolan-2-one of the formula (II) (50 g, 0.26 moles) was dissolved in methanol (150 ml) and water (225 ml) and cooled to 5-10° C., then conc. HCl (100 ml) was added dropwise to avoid temperature rise, and the mixture was stirred for 10 minutes. To this mixture sodium nitrite (36 g, 0.52 moles) solution in water (150 ml) was added slowly at −5 to 0° C. After addition, the solution was maintained under stirring at the same temp for 30 minutes.

Part B:

In a separate vessel, ethyl-2-acetyl-5-(1,3-dioxo-2,3-dihydro-1H-2-isoindolyl) pentanoate of the formula (III) (86.64 g, 0.27 moles) was dissolved in methanol (860 ml) an sodium acetate (170.60 g, 2.08 moles) was added; the mixture was stirred at room temperature for one hour and cooled to 0-5° C. To this mixture was slowly added the Part A mixture. After the addition, the mixture was stirred for 2-3 hours at room temperature. Progress of the reaction was monitored by TLC using ethyl acetate as the mobile phase.

The reaction mass was extracted with dichloromethane (250 ml×3 times). The combined dichloromethane layer was washed with water (100 ml×2 times), dried and concentrated. The crude residue (containing the hydrazone intermediate) was proceeded for cyclization to form the indole moiety.

The above residue (210 g) was dissolved in 100 ml of ethanol, heated to form a transferable mass and, under refluxing conditions, 450 ml ethanolic HCl (18.0%) were added. The mixture was then further refluxed for 12 hours. The reaction mass was cooled to room temperature and the obtained solid was filtered, and washed with methanol.

Product:

Net weight: 82 g Yield: 68% Purity: 98.00% (R=C₂H₅) Melting range: 200-205° C. ¹H-NMR (DMSO-d₆, 200 MHz): δ 1.40 (t, 3H), 2.62-2.8 (m, 2H), 2.83-2.98 (m, 2H), 3.38-3.42 (m, 2H), 3.92 (m, 2H), 4.10 (m, 1H), 4.26 (q, 2H), 7.16 (d, 1H, J=8.4 Hz), 7.38 (d, 1H, J=8.4 Hz), 7.62 (s, 1H), 7.78 (br, 4H), 11.51 (br, 1H, exchangeable). ¹³C-NMR (DMSO-d₆, 200 MHz): δ 14.138, 23.333, 38.271, 40.775, 51.456, 53.072, 60.272, 68.071, 112.589, 118.810, 119.052, 119.781, 122.861, 123.718, 123.984, 126.609, 127.557, 127.640, 131.502, 134.263, 135.227, 158.646, 161.613, 162.969, 167.667. MS: m/z 462 (100%, M+1).

### EXAMPLE 10

### Ethyl-3-[2-(1,3-dioxo-2,3-dihydro-1H-2-isoindoleyl)ethyl]-5-[(4S)-2-oxo-1,3-oxazolan-4-ylmethyl]-1H-2-indole carboxylate (VII)

Part A:

(4S)-4-(4-aminobenzyl)-1,3-oxazolan-2-one of the formula (II) (10 g, 0.052 moles) was dissolved in methanol (30 ml) and water (45 ml) and cooled to 5-10° C., then concentrated HCl (13.6 ml) was added dropwise to avoiding heating and the mixture was stirred for 10 minutes. To this mixture, sodium nitrite (4.5 g, 0.065 moles) solution in water (30 ml) was added slowly at −5 to 0° C. After the addition the solution was maintained under stirring at the same temperature for 30 minutes.

Part B:

In a separate vessel, alkyl-2-acetyl-5-(1,3-dioxo-2,3-dihydro-1H-2-isoindolyl) pentanoate of the formula (III) (17.32 g, 0.0546 moles) was dissolved in methanol (170 ml), sodium acetate (17 g, 0.21 moles) was added and the mixture was stirred at room temperature for one hour, cooled to 0-5° C. and the Part A mixture was added slowly. After the addition, the mixture was stirred for 2-3 hours at room temperature. Progress of the reaction was monitored by TLC, using ethyl acetate as the mobile phase.

The reaction mass was extracted with dichloromethane (2.5 L×3 times). The combined dichloromethane layer was washed with water (1.25 L×2 times), dried, and concentrated. The crude residue (containing the hydrazone intermediate) was proceeded for cyclization to form the indole moiety.

The above residue (30 g) was dissolved in 15 ml methanol, heated to form a transferable mass and, under refluxing conditions, 60 L Isopropanol-HCl (12%) was added. The reaction mixture was further refluxed for 12 hours. The reaction mass was then cooled to room temperature and the produced solid was filtered and washed with methanol.

Product:

Net weight: 14.5 g Yield: 60% Purity: 95.49% (R=C₂H₅ is 86.44%, R=Isopropyl is 9.05%) Melting range: 190-195° C. ¹H-NMR (DMSO-d₆, 200 MHz): δ 1.07 (d, O-ⁱPr), 1.35 (t, 3H), 2.6-2.9 (m, 2H), 3.3-3.4 (m, 2H), 3.8-3.9 (m, 4H), 4.08 (m, 1H), 4.21 (q, 2H), 7.04 (d, 1H, J=8.4 Hz), 7.35 (d, 1H, J=8.4 Hz), 7.38 (s, 1H, J=8.4 Hz), 7.78 (br, 4H), 11.51 (br, 1H, exchangeable). MS: m/z 462 (100%, M+1, Et ester), 477 (M+1 ⁱPr ester).

### EXAMPLE 11

### Ethyl 3-(2-dimethyl amino ethyl)-5-[(4S)-2-oxo-1,3-oxazolan-4-yl methyl]-1H-2-indole carboxylate (IX) (Via In Situ Intermediate VIII)

Ethyl-3-[2-(1,3-dioxo-2,3-dihydro-1H-2-isoindoleyl)ethyl]-5-[(4S)-2-oxo-1,3-oxazolan-4-ylmethyl]-1H-2-indole carboxylate of the formula (VII) (5 g, 10.85×10⁻³ moles) was added to 40 ml methanol at room temperature with stirring. 15 ml of a solution (10%) of methylamine in methanol was added dropwise in three portions under stirring at 25-30° C., and maintained under stirring at the same temperature for 4.5 hours. TLC was used to determine the reaction completion.

After the reaction was completed, also indicated by formation of a clear reaction mass, the reaction mass was neutralized by acetic acid to pH 6.5 to 7.0, 1.14 ml (1.69 g, 0.028 mole) of acetic acid were added and the mixture was cooled to 0-5° C. Then there were added 1.2 g (0.019 mole) of sodium cyanoborohydride. A mixture of 2.95 ml 37% aqueous solution of formaldehyde (1.09 g, 0.036 moles) and 10 ml methanol was added to the reaction mixture in a dropwise fashion at 0-5° C., under stirring. The reaction mixture was stirred at the same temperature for another 60-90 minutes and then monitored by TLC using 30% methanol and 1.5% NH₃ in dichloromethane as the mobile phase. TLC showed the completion of reaction, as the starting material disappeared.

To the reaction mixture a solution of potassium carbonate in water (2.99 g in 10 ml) was added at 0-5° C. and the mixture was then extracted with 100 ml×3 times of ethyl acetate. All ethyl acetate layers were combined, washed with water followed by brine solution and evaporated to dryness to yield ethyl 3-(2-dimethyl amino ethyl)-5-[(4S)-2-oxo-1,3-oxazolan-4-yl methyl]-1H-2-indole carboxylate.

Product:

Net weight: 1.4 g Yield: 36% Melting range: 49-55° C. IR: 3384 (br), 2934 (br), 1745, 1708, 1546, 1461, 1398, 1331, 1240, 1090, 1023 cm⁻¹. ¹H-NMR (DMSO-d₆, 200 MHz): δ 1.39 (t, 3H), 2.62-3.0 (m, 8H), 3.18-3.27 (br, 2H), 3.42-3.48 (br, 2H), 3.86 (s, —OCH₃), 3.89-4.19 (m, 2H), 4.21-4.42 (m, 2H), 7.19 (d, 1H, J=8.4 Hz), 7.40 (d, 1H, J=8.4 Hz), 7.67 (s, 1H, J=8.4 Hz), 7.84 (s, 1H, exchangeable), 10.84 (br, 1H, exchangeable), 11.62-11.79 (br, 1H, exchangeable). MS: m/z 360 (M+1; Et ester, 100%), 346 (M+1; Me ester).

### EXAMPLE 12

### 3-[2-(1,3-dioxo-2,3,3a,7a-tetrahydro-1H-2-isoindolyl)-ethyl]-5-[(4S)-2-oxo-1,3-oxazolan-4-ylmethyl]-1H-2-indolecarboxylic acid (X)

A solution of sodium carbonate (11.5 g, 0.1085 mole) in water (400 ml) was added to 50 g (0.1085 mole) ethyl-3-[2-(1,3-dioxo-2,3-dihydro-1H-2-isoindoleyl) ethyl]-5-[(4S)-2-oxo-1,3-oxazolan-4-ylmethyl]-1H-2-indole carboxylate (VII) and the mixture was refluxed for 2 hours to obtain a clear solution.

The mixture was acidified to pH 2 with concentrated HCl at 15-20° C. and extracted with ethyl acetate vigorously. The ethyl acetate layer was washed with water to a neutral pH, dried over sodium sulfate, and evaporated completely to yield the crude product (40 g, 85%).

To 20 g of this crude, 200 ml of xylene (LR grade) and 10 ml of glacial acetic acid were added and refluxed at 138-140° C. for 15-17 hours. The mixture was then cooled and the free flowing solid was then filtered, washed with ethyl acetate to remove xylene, and dried.

Product:

Net weight: 12 g Yield: 51% Melting range: 274-276° C. IR (KBr): 3391 (br), 2925, 1771, 1713, 1579, 1553, 1397, 1358, 1220, 1179, 1102, 1017, 716 cm⁻¹. ¹H-NMR (DMSO-d₆, 400 MHz): δ 2.62 (dd, J=13, 8, 1H), 2.77 (dd, J=13, 5, 1H), 3.37 (t, 1H), 3.80 (m, 2H), 3.86 (t, 2H), 3.87 (t, 1H), 4.04 (t, 1H), 7.03 (dd, J=8, 2, 1H), 7.27 (d, J=8, 1H), 7.31 (br, s, 1H), 7.72 (NH), 7.77 (br, s, 2H), 7.77, br, s, 2H), 11.4 (NH), 13.00 (OH). ¹³C-NMR (DMSO-d₆, 400 MHz): δ 23.24, 38.22, 40.63, 53.16, 68.11, 112.53, 118.44, 119.66, 122.85, 124.96, 126.21, 127.39, 127.80, 131.52, 134.21, 134.99, 158.70, 163.22, 167.77. MS (Electronspray,+ve mode): m/z 434 (M+H)⁺, 456 (M+Na)⁺, 867 (2M+1)⁺, 884 (2M+NH3)⁺. MS (Electronspray, −ve mode): m/z 432 (M−H)⁻, 866 (2M−H)⁻.

1. A process for preparing zolmitriptan, comprising the steps:
(a) forming a diazo salt having formula (IV) from (4S)-4-(4-aminobenzyl)-1,3-oxazolan-2-one, 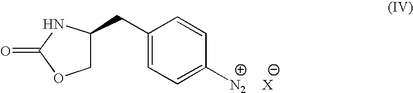
where X represents a halogen;
(b) forming an enol derivative having formula (V) from alkyl-2-acetyl-5-(1,3-dioxo-2,3-dihydro-1H-2-isoindolyl) pentanoate, 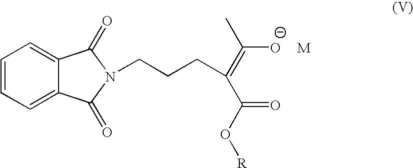
where R represents a straight or branched chain C₁-C₄ alkyl group and M represents an alkali metal;
(c) coupling diazo salt (IV) and enol derivative (V) to form a hydrazone having formula (VI) 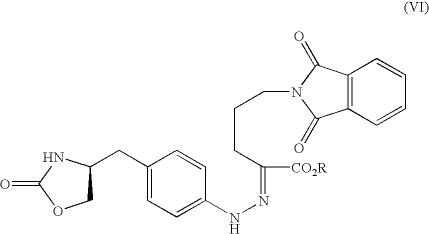
where R represents a straight or branched chain C₁-C₄ alkyl group;
(d) a cyclization of hydrazone (VI) to form an indole derivative having formula (VII) 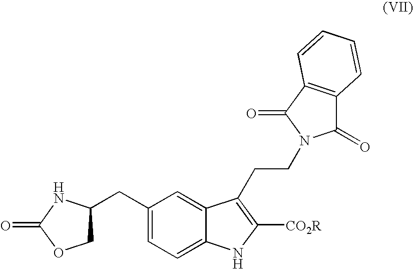
where R represents a straight or branched chain C₁-C₄ alkyl group;
(e) deprotecing the phthalimide moiety of (VII) to form a primary amine having formula (VIII), 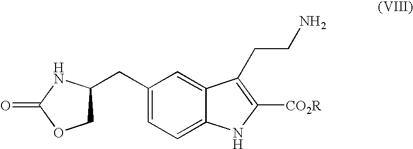
where R represents a straight or branched chain C₁-C₄ alkyl group;
(f) N,N-dimethylating primary amine (VIII) to form a tertiary amine having formula (IX), 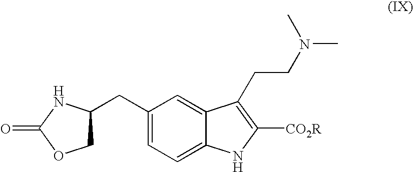
where R represents a straight or branched chain C₁-C₄ alkyl group; and
(g) decarboxylating tertiary amine (IX) to form zolmitriptan.

2. The process of claim 1, wherein the hydrazone (VI) is not isolated, but is reacted in situ in step (d).

3. The process of claim 1, wherein the tertiary amine (IX) is not isolated, but is reacted in situ in step (g).

4. The process of claim 1, wherein the primary amine (VIII) is treated with an acid to form an amine salt, before step (f).

5. A process for preparing zolmitriptan, comprising the steps:
(i) hydrolyzing an an indole derivative having formula (VII), 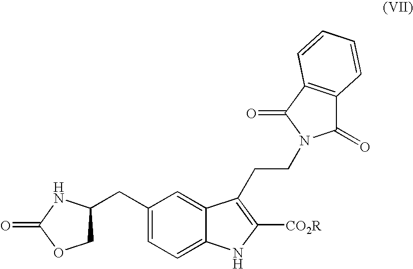
to form an acid having formula (X), 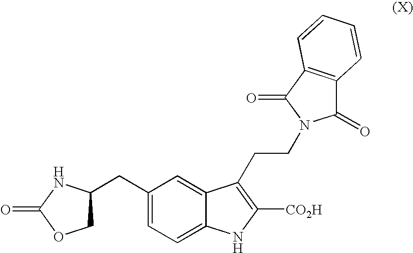
(ii) decarboxylating the acid (X) to form an isoindoledione having formula (XI), 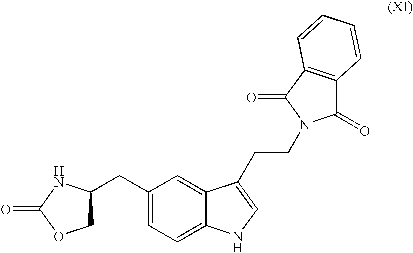
(iii) deprotecting the phthalimide moiety of the isoindoledione (XI) to form an oxazolan-2-one having formula (XII), 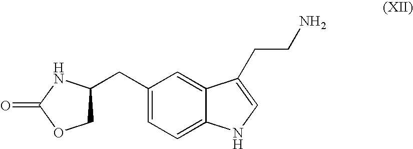
and
(iv) N,N-dimethylating the oxazolan-2-one (XII) to form zolmitriptan.

6. The process of claim 5, wherein the acid having formula (X) is not isolated, but is reacted in situ in step (ii).

7. The process of claim 5, wherein the isoindoledione having formula (XI) is not isolated, but is reacted in situ in step (iii).

8. The process of claim 5, wherein the isoindoledione having formula (XI) and acid having formula (X) are not isolated, but are respectively reacted in situ in steps (ii) and (iii).

9. A compound having structural formula (VI), 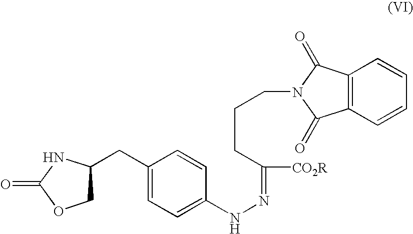
where R represents a straight or branched chain C₁-C₄ alkyl group.

10. A compound having structural formula (VII), 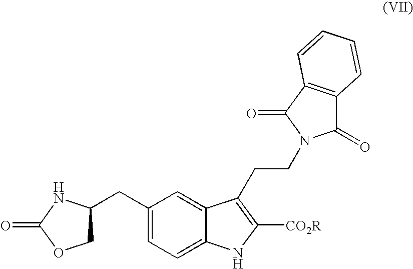
where R represents a straight or branched chain C₁-C₄ alkyl group.

11. A compound having structural formula (VIII), 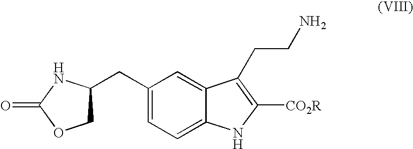
where R represents a straight or branched chain C₁-C₄ alkyl group, or an amine salt of the compound.

12. A compound having structural formula (X),

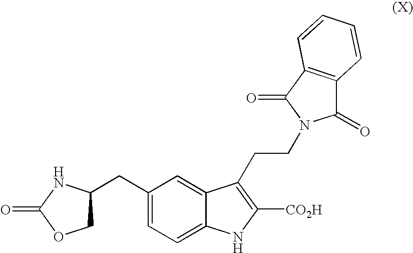

13. A compound having structural formula (XI):

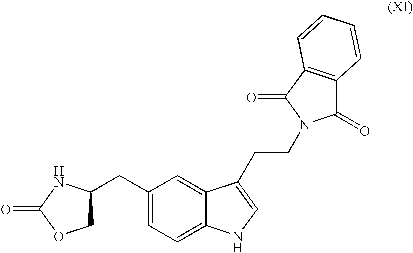

14. A process for purifying optically pure zolmitriptan, comprising:
(1) dissolving zolmitriptan in isopropyl alcohol at an elevated temperature;
(2) cooling the solution to temperatures between about 20° C. and about 30° C.;
(3) gradually adding n-Heptane to the mixture, with stirring at temperatures about 20° C. to about 30° C.; and
(4) isolating zolmitriptan.

15. The process of claim 14, wherein steps (1) through (4) are repeated at least once to minimize zolmitriptan impurity content.
